(19) United States
(12) Patent Application Publication
(10) Pub. No.: US 2024/0082509 A1
(43) Pub. Date: Mar. 14, 2024

(54) LOW DEAD SPACE NEEDLE

(71) Applicants: Sophie Anna Louise RUSSELL, Le Pradet (FR); Ido TOPEL, London (GB)

(72) Inventors: Sophie Anna Louise RUSSELL, Le Pradet (FR); Ido TOPEL, London (GB)

(21) Appl. No.: 17/942,145

(22) Filed: Sep. 11, 2022

(51) Int. Cl. A61M 5/32; A61M 5/34

(52) U.S. Cl. CPC A61M 5/3293; A61M 5/343; A61M 5/348; A61M 2205/02

(57) ABSTRACT

There is provided a needle hub assembly for connecting to a barrel with plunger for assembling a syringe, comprising: a cylindrical depression in a proximal end portion of the needle hub assembly, the cylindrical depression having a flat bottom perpendicular to a longitudinal axis of the needle hub assembly, wherein the cylindrical depression is shaped for connecting to a nozzle of the barrel and for contacting between the flat bottom and a distal end of the nozzle surrounding an opening of the nozzle when the nozzle is disposed within the cylindrical depression, a lumen extending from the flat bottom of the cylindrical depression to a distal end portion of the needle hub assembly, and a cannula having a proximal cannula portion located within the lumen and a distal cannula portion extending distally from the distal end portion of the needle hub assembly.

### FIELD AND BACKGROUND OF THE INVENTION

The present invention, in some embodiments thereof, relates to a medical device and, more particularly, but not exclusively, to a low dead space needles.

Syringes are routinely used in medicine to administer an injection such as a vaccine and/or an anesthetic (e.g., to repair a cut, treat a tooth). Syringes may also be used to infuse intravenous therapy, for example, adding a controlled amount of a drug to a saline IV drip. Syringes are commonly disposable, and used in vast quantities on a daily basis.

### SUMMARY OF THE INVENTION

According to a first aspect, a needle hub assembly for connecting to a barrel with plunger for assembling a syringe, comprises: a cylindrical depression in a proximal end portion of the needle hub assembly, the cylindrical depression having a flat bottom perpendicular to a longitudinal axis of the needle hub assembly, wherein the cylindrical depression is shaped for connecting to a nozzle of the barrel and for contacting between the flat bottom and a distal end of the nozzle surrounding an opening of the nozzle when the nozzle is disposed within the cylindrical depression, a lumen extending from the flat bottom of the cylindrical depression to a distal end portion of the needle hub assembly, and a cannula having a proximal cannula portion located within the lumen and a distal cannula portion extending distally from the distal end portion of the needle hub assembly.

In a further implementation form of the first aspect, a depth of the cylindrical depression measured parallel to the longitudinal axis is greater than 7 millimeters (mm).

In a further implementation form of the first aspect, an inner surface of the cylindrical depression extending from the flat bottom to a depth 120 away from an edge of the proximal end portion, is sized and shaped to contact and fluidly seal an exterior surface of the nozzle of the barrel when the barrel is disposed within the cylindrical depression.

In a further implementation form of the first aspect, the inner surface of the cylindrical depression is sized and/or shaped for compliance with ISO 80369-7.

In a further implementation form of the first aspect, the needle hub assembly is sized and/or shaped for compliance with ISO 594-1.

In a further implementation form of the first aspect, the inner surface of the cylindrical depression is set to a 6% Luer tap.

In a further implementation form of the first aspect, further comprising a pair of flares extending outwardly from opposite locations of an external surface of an end of the proximal end portion of the needle hub assembly, wherein a length from an end of a first flare to an end of a second flare perpendicular to the longitudinal axis is about 7.8 millimeters and a diameter of the first end portion excluding the pair of flares is about 6.7 mm.

In a further implementation form of the first aspect, the cylindrical depression is sized and shaped for fitting Luer slip and Luer Lock Tip devices.

In a further implementation form of the first aspect, the cylindrical depression and cannula are sized and shaped to enclose a volume of about 0.0052 milliliters (mL) when a low dead space plunger is fully plunged into the barrel connected to the cylindrical depression.

In a further implementation form of the first aspect, the lumen comprises a proximal lumen portion having a constant internal diameter along a length thereof and a distal lumen portion having a tapered diameter increasing in diameter from a junction with the proximal lumen portion towards an opening at the distal end portion of the needle hub assembly, the proximal lumen portion having an opening into the cylindrical depression.

In a further implementation form of the first aspect, the proximal cannula portion is disposed within the distal lumen portion, wherein at the junction the cannula is in continuity with the distal lumen portion, wherein the cannula has a constant inner diameter along a length thereof matching a constant inner diameter of the distal lumen portion, thereby forming a fluid flowing channel with continuous constant inner diameter for fluid flowing from the barrel via the cylindrical depression via the proximal lumen portion and through the cannula.

In a further implementation form of the first aspect, the cannula is not disposed within the proximal lumen portion.

In a further implementation form of the first aspect, an adhesive is disposed within a space formed between an exterior surface of the proximal cannula portion disposed within the lumen and an interior surface of the tapered distal lumen portion.

In a further implementation form of the first aspect, the adhesive extends externally from the space, forming a cone and/or convex shape having a wider base connected to an end surface of the distal end portion of the needle hub assembly perpendicular to the longitudinal axis, wherein the cannula extends through an interior of the cone and/or convex shape of the extending adhesive.

In a further implementation form of the first aspect, further comprising a support element having a wider base connected to an end surface of the distal end portion of the needle hub assembly perpendicular to the longitudinal axis, wherein the cannula extends through an interior of the support element.

In a further implementation form of the first aspect, the support element comprises an adhesive that supports a portion of the cannula extending past the distal end portion of the needle hub assembly by connecting the cannula extending through the interior of the support element with the end surface of the distal end portion of the needle hub assembly.

In a further implementation form of the first aspect, the cannula is made using medical silicon.

In a further implementation form of the first aspect, the cannula is lubricated by medical silicon fluid.

In a further implementation form of the first aspect, the cannula is made from materials compliant with ISO 9626.

In a further implementation form of the first aspect, a bevel angle of the cannula comprises a triple point.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWING(S)

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is a schematic to help understand improvement to the technical field of syringes obtained by the needle hub assembly described herein in comparison to other known syringes, in accordance with some embodiments of the present invention;

FIG. 2 is a schematic depicting a cross sectional slice of a needle hub assembly for connecting to a barrel with plunger for assembling a syringe that has a low retained volume, in accordance with some embodiments of the present invention;

FIG. 3 is a schematic depicting a side perspective view of needle hub assembly described herein and/or with reference to FIG. 2, in accordance with some embodiments of the present invention;

FIG. 4 is a schematic depicting a face-on/top view perspective view of needle hub assembly described herein and/or with reference to FIG. 2, in accordance with some embodiments of the present invention;

FIG. 5 is a schematic depicting a perspective view of needle hub assembly showing cannula described herein and/or with reference to FIG. 2, in accordance with some embodiments of the present invention;

FIG. 6 is the schematic of the needle hub assembly depicted in FIG. 2, indicating exemplary dimensions that comply with ISO standards, in accordance with some embodiments of the present invention;

FIG. 7 is the schematic of the needle hub assembly depicted in FIG. 3, indicating exemplary dimensions that comply with ISO standards, in accordance with some embodiments of the present invention; and

FIG. 8 is the schematic of the needle hub assembly depicted in FIG. 4, indicating exemplary dimensions that comply with ISO standards, in accordance with some embodiments of the present invention.

### DESCRIPTION OF SPECIFIC EMBODIMENTS OF THE INVENTION

The present invention, in some embodiments thereof, relates to a medical devices and, more particularly, but not exclusively, to a low dead space needles.

An aspect of some embodiments of the present invention relates to a needle hub assembly designed to connect to a barrel with plunger for assembling a syringe that has low dead space of remaining fluid therein after the plunger has been fully pushed into the barrel. The needle hub assembly is sized and/or shaped to fit standard barrels and plungers, for example, according to the International Organization for Standardization (ISO). The needle hub assembly includes a cylindrical depression in a proximal end portion thereof. The cylindrical depression has a flat bottom. The flat bottom is arranged perpendicular to a longitudinal axis of the needle hub assembly. The cylindrical depression is sized and/or shaped for contacting an exterior surface of a nozzle of the barrel, optionally according to ISO guidelines. The cylindrical depression is sized and/or shaped to form contact between the flat bottom and a surface in proximity to the opening of the nozzle, and between an interior surface of the cylindrical depression and an exterior surface of the nozzle along a longitudinal axis of the nozzle. The contact between the cylindrical depression and the nozzle is designed to be fluid tight, such that fluid existing from an opening in the nozzle is forced into an opening of a lumen in the flat bottom without having a space for existing between the cylindrical depression and the nozzle. The lumen extends from the flat bottom of the cylindrical depression to a distal end portion of the needle hub assembly. A cannula, for example, a needle, having a proximal cannula portion is located within the lumen. A distal cannula portion of the cannula extends distally from the distal end portion of the needle hub assembly. The amount of fluid remaining in the cannula, the portion of the lumen that that does include the cannula, and a space formed by the nozzle, flat bottom of the cylindrical depression, and interior walls of the cylindrical depression, after the plunger has been fully pressed to inject fluid contents of the barrel, is designed to be low, for example, about 5 microliters, which may be lower than other syringe designs, in particular other syringes assembled from other needle hubs that are compliant with ISO guidelines.

At least some implementations of the needle hub assemble described herein addresses the technical problem of reducing wasted fluid during injections. After performing an injection using a syringe, previously designed needles, such a standard needles, retain a portion of the fluid therein. That is out of a volume of fluid which is loaded into the barrel, the entire volume is not injected. A certain portion of the volume is injected and another portion is retained. Now, when considering the large number of syringes that are used (e.g., on a daily basis), the sum of the retained portions of the vast number of syringes becomes significant. For example, vaccinations, such as for corona virus (i.e., COVID-19), are routinely performed on a large number of people, which may reach millions, or hundreds of millions, or billions of used syringes. Reducing the amount of fluid retained in each syringe may add up to significant savings. The savings may be especially important if the fluid (e.g., vaccine) is in short supply and/or expensive (e.g., botox injections). For example, reducing the amount of fluid retained in each syringe may enable loading 6 vaccine syringes from a single vial of vaccine (e.g., for corona virus, i.e., COVID-19) in comparison to loading vaccine syringes from the single vial using standard syringes, which enables vaccinating a significantly larger number of people using the same supply of vials.

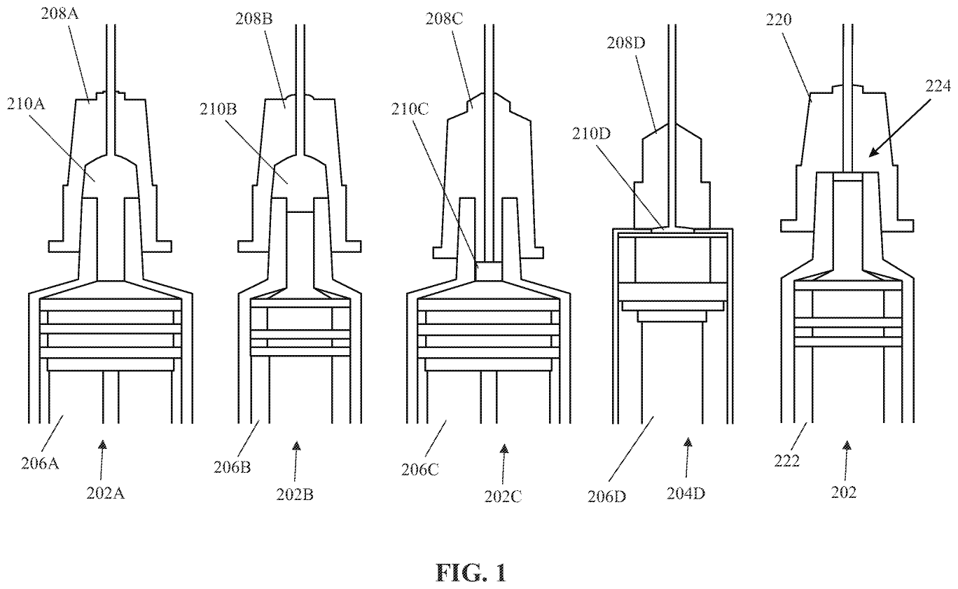

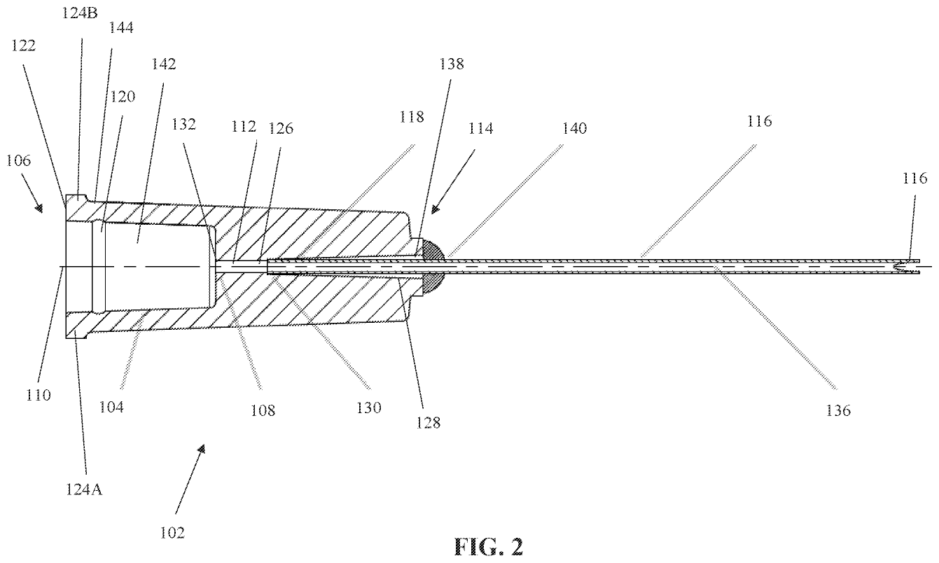

Reference is now made to FIG. 1, which is a schematic to help understand improvement to the technical field of syringes obtained by the needle hub assembly 102 (e.g., as described with reference to FIG. 2) in comparison to other known syringes 202A-D, in accordance with some embodiments of the present invention.

Syringe 202A is a standard barrel and plunger 206A connected to a standard needle hub 208A, which retains a very large volume of fluid 210A therein, that cannot be injected, and therefore lost.

Syringe 202B is a low dead space barrel and plunger 206B connected to a standard needle hub 208B, which still retains a large volume of fluid 210B therein, although it may be less than volume of fluid 210A retained by syringe 202A.

Syringe 202C is a standard barrel and plunger 206C connected to a needle hub 208C designed to be low dead space. Needle hub 208C may be as described herein, and/or other known designs. A smaller volume of fluid 210C is retained, which is less than volume of fluid 210A retained by syringe 202A and less than volume of fluid 210B retained by syringe 202B.

Syringe 202D is a known special design that is an integrated barrel and plunger 206D fixed to a needle hub 208D. Since the entire syringe 202D is integrated and fixed, needle hub 208D is not a separate component and cannot be used with different separate barrel and plunger components. Although retained volume 210D is low, and may be lower than volumes 210A-C of syringes 210A-C, this comes at the cost of limiting the user to using this specific syringe. The user is required to obtain syringe 202D as a whole from a specific manufacturer. The user is not provided with the ability to select different barrel and plungers.

Moreover, another known design (not shown) includes barrel and plunger components which are separate from the needle hub. The needle hub is designed to retain a low volume of fluid after plunger has been fully pressed. Although potentially the user may select the barrel and plunger to connect to the needle hub, the needle hub is not designed to comply with standards such as ISO guidelines, and therefore, the barrel and plunger that are able to connect to the needle hub are specialize products available only from specific manufacturers, which severely limits the ability of the user to use standard components from different manufactures. Even in implementations in which standard barrel and plungers may fit the specialized needle hub, the resulting syringe does not comply with standards such as ISO guidelines, and may experience other failures, such as stability issues, leakage of fluid, and the like.

In contrast, as described herein, syringe 202 is assembled from a needle hub assembly 220 described herein, which is sized and/or shaped to fit standard barrels and plunger 222 components. The improvement is in the flat bottom of a cylindrical depression of the needle hub assembly 220, and the shape and/or size of the interior walls of the cylindrical depression, that minimize space between the interior of the cylindrical depression when the nozzle of the barrel 222 is located within the cylindrical depression. This design reduces an amount of retained fluid 224, which may be less than fluid 210A-D of syringes 202A-D. The amount of retained fluid 224 is especially reduced when a standard low dead weight plunger is used.

For example, standard needle hubs retain about 0.0192 milliliter (mL) or about 19 microliters. The needle hub assemble described herein retains about 0.0052 mL or about 5 microliters.

It is noted that in some prior designs, a hub connector of the needle hub designed to connect to a barrel and plunger is not flat, but includes an elevation (e.g., bump). This elevation prevents the nozzle from contacting the bottom surface of the hub connector. When the nozzle contacts the elevation, a space is formed between the nozzle and the bottom surface (i.e., the rest of the hub connector in proximity to the elevation, but not part of the elevation) which retains fluid after the plunger is maximally displaced.

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways.

As used herein, the terms distal and proximal are with reference to a user holding an assembled syringe in a position ready for injection (e.g., into a subject). Components of the syringe closer to the user (and further away from the subject) are termed proximal, and components further away from the user (and closer to the subject) are termed distal.

As used herein, ISO standards are current and/or past standards. It is to be understand that future ISO standards may be defined, and/or new versions and/or updates of existing standards may be created, and the term ISO standard is meant to cover past, current, and future relevant ISO standards.

Reference is now made to FIG. 2, which is a schematic depicting a cross sectional slice of a needle hub assembly 102 for connecting to a barrel with plunger for assembling a syringe that has a low retained volume after the plunger is maximally displaced (i.e., pressed) to expel fluid within the barrel, in accordance with some embodiments of the present invention.

The following is a list of elements used in labelling FIG. 2:
102 Needle hub assembly
104 cylindrical depression
106 proximal end of needle hub assembly
108 flat bottom
110 longitudinal axis
112 lumen
114 distal end of the needle hub
116 cannula
118 proximal cannula portion
120 depth
122 edge of proximal end
124A-B flares
126 proximal lumen portion
128 distal lumen portion
130 junction of proximal lumen portion and distal lumen portion
132 opening into cylindrical depression
134 distal cannula portion
136 fluid flow line
138 space formed between exterior surface of proximal cannula portion and interior surface of tapered distal lumen portion
140 adhesive/support element
142 inner surface of cylindrical depression
144 external surface

Needle hub assembly 102 includes a cylindrical depression 104 in a proximal end portion 106 thereof. Cylindrical depression 104 is a hub connector component, designed to connect to a hub. Cylindrical depression has a flat bottom 108. Flat bottom 108 is arranged perpendicular to a longitudinal axis 110 of needle hub assembly 102. The flat bottom 108 enables contact between a surface proximal to an opening of a nozzle of the barrel when the barrel is connected to need hub assembly 102 by being disposed within cylindrical depression 104. Cylindrical depression 104 is sized and/or shaped for contacting between an interior surface 142 of the cylindrical depression 104 and an exterior surface of the nozzle along a longitudinal axis of the nozzle. Interior surface 142 of the cylindrical depression extends from the flat bottom 108 to a depth 120 away from an edge 122 of the proximal end portion 106. The interior surface located at the depth 120 region may include elements designed to secure the nozzle, for example, a screw. The contact between the cylindrical depression 104 and the nozzle is designed to be sealed, i.e., fluid tight, such that fluid existing from an opening in the nozzle is entirely forced into an opening of a lumen 112 in the flat bottom 108 without having a space for existing between the cylindrical depression 104 and the nozzle.

Lumen 112 extends from the flat bottom 108 of the cylindrical depression 104 to a distal end portion 114 of the needle hub assembly. A cannula 116, for example, a needle, having a proximal cannula portion 118 is located within the lumen, in particular within a distal lumen portion 128. A distal cannula portion 134 of the cannula 116 extends distally from the distal end portion 106 of the needle hub assembly 102.

Cylindrical depression 104, lumen 112 and cannula 116 are sized and shaped to enclose a volume of about 0.0052 milliliters (mL) when a low dead space plunger is fully plunged into the barrel connected to the cylindrical depression 104. The amount of fluid remaining in cannula 116, a proximal lumen portion 126 portion of the lumen (i.e., that that does include the cannula), and a space formed by the nozzle, flat bottom 108 of the cylindrical depression 104, and interior walls 142 of the cylindrical depression, after the plunger has been fully displaced (i.e., pressed) to inject fluid contents of the barrel, is designed to be low, for example, about 0.0052 mL or about 5 microliters, which may be lower than other syringe designs, in particular other syringes assembled from other needle hubs that are compliant with ISO guidelines, as described herein. This provides a savings of up to about 0.014 mL or about 14 microliters of fluid (e.g., vaccine, medicine, botox, anesthetic) in comparison to using standard hubs, barrels, and plungers, for example, as experimentally verified by Inventors and as described with reference to the “Examples” section below.

Dimensions and/or sizing of needle hub assembly 102 are selected to be in compliance with standards, for example, ISO standards, such as ISO 594-1. This enables needle hub assembly 102 to connect to any barrels and plungers that are also compliant with the same standards, providing the user with the ability to select off the shelf barrels and/or plungers, for example, of different sizes, shapes, and/or manufactures. For example a depth of the cylindrical depression 104 measured from edge 122 of proximal end 106 of the needle hub assembly, parallel to the longitudinal axis 110 may be greater than 7 millimeters (mm). In some implementations, the depth is equal to 7 mm.

It is noted that in another prior known hub design, the length of a barrel connector of a hub designed to connect to a barrel is non-compliant with ISO guidelines, or minimally compliant with ISO guidelines (e.g., having a size at the limit of the ISO guidelines).

Optionally, dimensions of inner surface 142 of cylindrical depression 104 are selected for compliance with guidelines, optionally ISO guidelines, for example, ISO 80369-7. Dimensions of inner surface 142 may be set to a 6% Luer tap and/or sized and/or shaped Dimensions of a bottom surface.

Optionally, dimensions of hub 102 and/or cylindrical depression 104 are selected to be compliance with standards, optionally ISO guidelines such as 7864, for fitting Luer Slip and/or Luer Lock Tip barrels, syringes, and/or other medical devices with compliant tip connectors.

Needle hub assembly 102 may include a pair of flares (124A-B) extending outwardly from opposite locations of an external surface 144 of needle hub assembly 102, at a location corresponding to an end of proximal end portion 106 of the needle hub assembly 102, such as extending from edge 122 of proximal end portion 106. The dimensions of flares 124A-B and/or dimensions of external surface 144 of needle hub assembly 102 may be compliant with 7864 for fitting Luer Slip and/or Luer Lock Tip barrels, syringes, and/or other medical devices with compliant tip connectors. For example, a length from an end of a first flare 124A to an end of a second flare 124B perpendicular to the longitudinal axis 110 is about 7.8 millimeters and a diameter of the first end portion excluding the pair of flares 124A-B is about 6.7 mm. Lumen 112 includes a proximal lumen portion 126 and a distal lumen portion 128. Proximal lumen portion 126 has a constant internal diameter along a length thereof. One end of proximal lumen portion 126 has opening 132 into flat bottom 108 of cylindrical depression 104. The opposite end of proximal lumen portion 126 connects to distal lumen portion 128 at a junction 130. Distal lumen portion 128 has a tapered diameter increasing in diameter from junction 130 with proximal lumen portion 126 towards an opening at the distal end portion 114 of the needle hub assembly 102.

Fluid flows along a flow flowing channel 136, substantially parallel to longitudinal axis 110, in response to plunger being displaced (i.e., pressed) when barrel is connected to needle hub assembly 102, for example, during injection of the fluid. Needle hub assembly 102 is designed to generate flowrate consistency of the flowing fluid in response the plunger being displaced, by having a constant internal diameter dimension along flow rate line 136, from opening 132 of flat bottom 108 of cylindrical depression 104 until fluid exits the tip of cannula 116.

Proximal cannula portion 118 is located within distal lumen portion 128. Cannula 116 is not disposed within proximal lumen portion 118. At junction 130, cannula 116 is in continuity with distal lumen portion 128. Cannula 116 has a constant inner diameter along a length thereof, that matches a constant inner diameter of distal lumen portion 128. The continuity of the constant inner diameters forms a fluid flowing channel 136 with continuous constant inner diameter for fluid flowing from the barrel via opening 132 of flat bottom 108 of cylindrical depression 104 via proximal lumen portion 126 and through cannula 116.

The constant inner diameter improves efficiency of the flowing fluid, for example, creating laminar flow, and/or reducing and/or avoiding turbulent flow which may create complications during injections and/or avoid unnecessary required applied force to depress the plunger.

Optionally, an adhesive is disposed within a space 138 formed between an exterior surface of proximal cannula portion 118 disposed within the lumen 112 (i.e., within the distal lumen portion 128) and an interior surface of the tapered distal lumen portion 128. The adhesive provides a strong bonding force between cannula 116 and needle hub assembly 102, for example, for preventing cannula 116 from detaching from needle hub assembly 102, and/or for preventing cannula 116 from bending.

Optionally, the adhesive extends externally from the space 138. The portion of the adhesive external to space 138, extending above distal end portion 114 of needle hub assembly, may form a cone and/or convex shaped support 140. The wider base of support element 140 is connected to the end surface of the distal end portion 114 of the needle hub assembly 102 perpendicular to the longitudinal axis 110. Cannula 116 extends through an interior of the support element 140. Alternatively, support element 140 is separate from the adhesive in space 138, for example, another adhesive separately applied, and/or a molding of distal end portion 114 of needle hub assembly into the convex and/or cone shape.

Support element 140 is positioned to support a portion of cannula 116 extending past the distal end portion 114 of the needle hub assembly 102 by supporting the portion of cannula 116 extending through the interior of support element 140 and/or by connecting the portion of cannula 116 extending through the interior of support element 140 with the end surface of the distal end portion 114 of the needle hub assembly 102.

Support element 140 is designed to provide additional support to cannula 116, to avoid and/or reduce risk of bending during injections.

Cannula 116 may be implemented as, for example, a needle (e.g., for injection into a subject) and/or tubing (e.g., connected to an IV drip).

Cannula 116 may be made, for example, using medical silicon, metal, and/or other medical grade materials. Cannula 116 may be made of materials that comply with a standard(s), such as ISO standard(s), for example, ISO 9626.

Cannula 116 may be lubricated by medical silicon fluid. The lubrication with medical silicon is designed to provide a smooth penetration force during injection into tissue of a subject.

A bevel angle 142 of cannula 116 may be a triple point, optionally that complies with a standard(s), such as ISO standard(s), for example, ISO 7864.

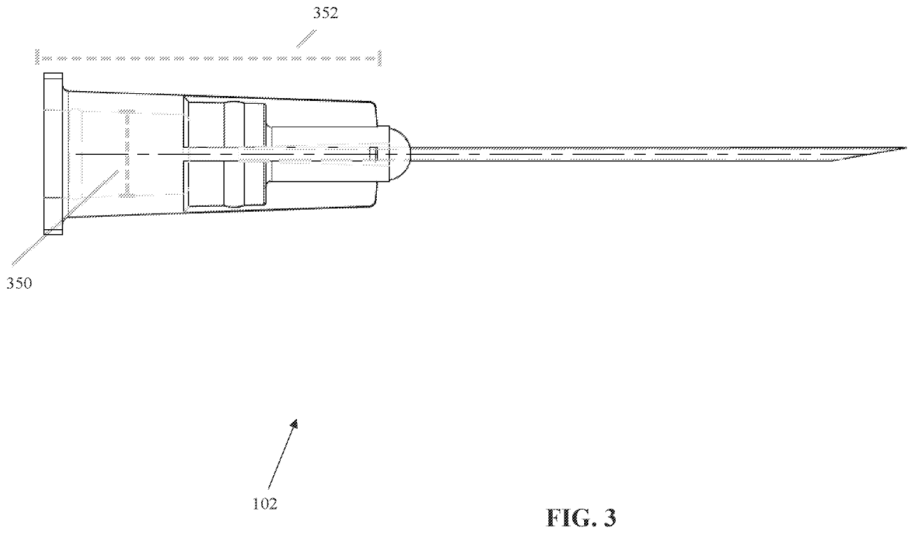

Reference is also made to FIG. 3, which is a schematic depicting a side perspective view of needle hub assembly 102 described herein and/or with reference to FIG. 2, in accordance with some embodiments of the present invention. Dimensions 350 of cylindrical depression 104 and/or dimensions 352 of needle hub assembly 102 are selected to comply with relevant ISO standards, as described herein.

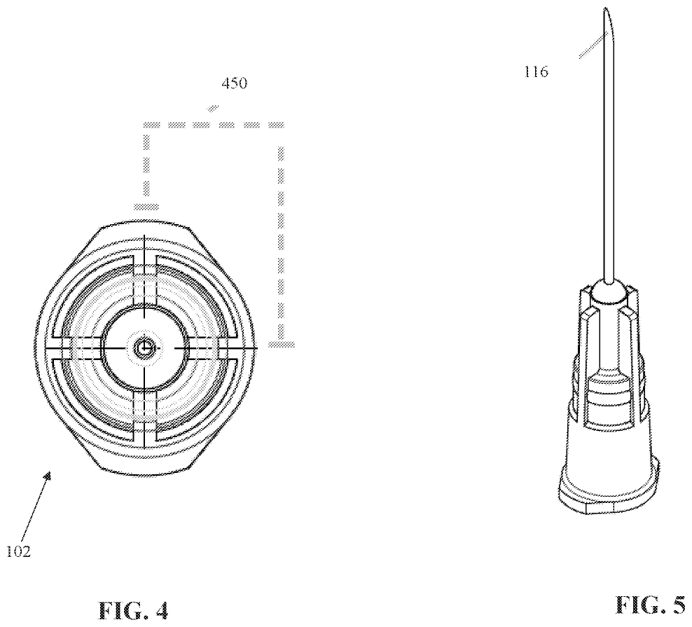

Reference is also made to FIG. 4, which is a schematic depicting a face-on/top view perspective view of needle hub assembly 102 described herein and/or with reference to FIG. 2, in accordance with some embodiments of the present invention. Dimensions 450 of needle hub assembly 102 are selected to comply with relevant ISO standards, as described herein.

Reference is now made to FIG. 5, which is a schematic depicting a perspective view of needle hub assembly 102 showing cannula 116 described herein and/or with reference to FIG. 2, in accordance with some embodiments of the present invention.

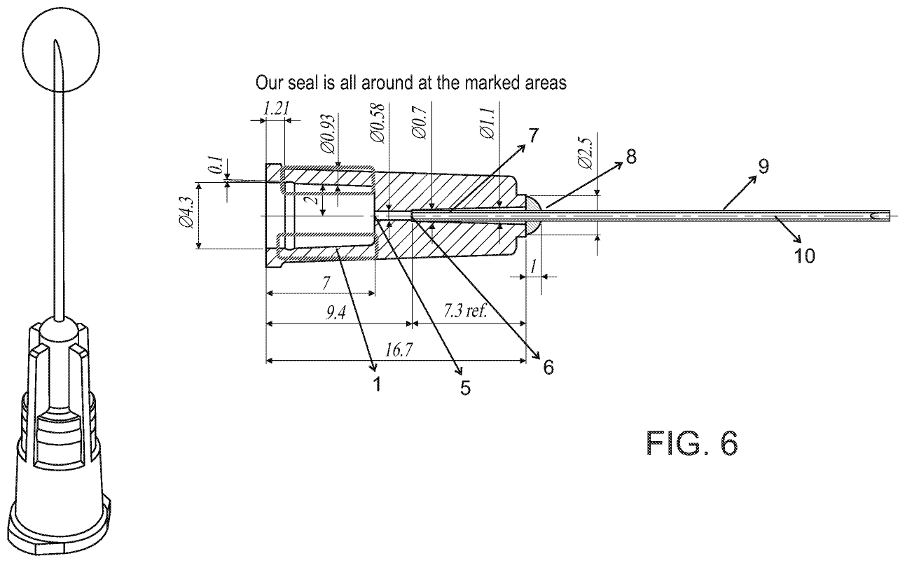

Reference is now made to FIG. 6, which is the schematic of needle hub assembly 102 depicted in FIG. 2, indicating exemplary dimensions that comply with ISO standards, in accordance with some embodiments of the present invention.

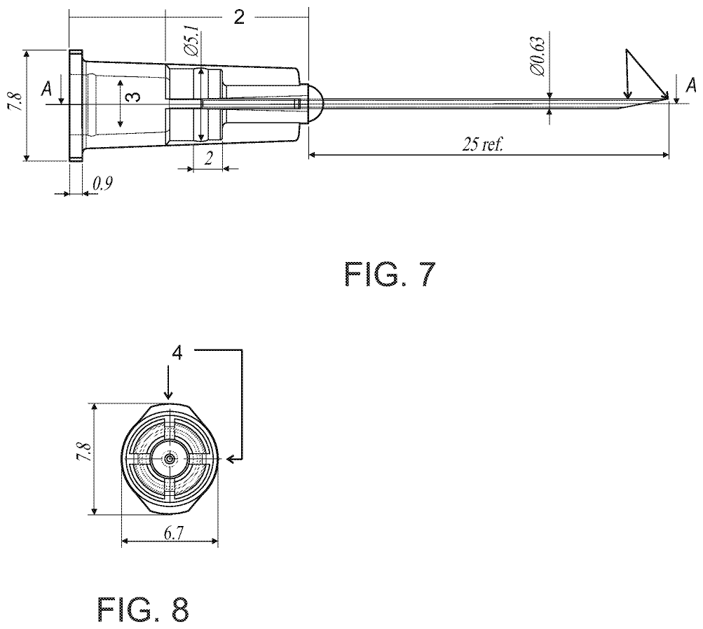

Reference is now made to FIG. 7, which is the schematic of needle hub assembly 102 depicted in FIG. 3, indicating exemplary dimensions that comply with ISO standards, in accordance with some embodiments of the present invention.

Reference is now made to FIG. 8, which is the schematic of needle hub assembly 102 depicted in FIG. 4, indicating exemplary dimensions that comply with ISO standards, in accordance with some embodiments of the present invention.

It is expected that during the life of a patent maturing from this application many relevant barrels, plungers, needles, syringes, and cannulas will be developed and the scope of the terms barrel, plunger, needle, syringe, and cannula are intended to include all such new technologies a priori.

As used herein the term “about” refers to ±10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion.

Inventors performed experiments to measure the amount of fluid retained by syringes assembled from needle hub assemblies based on embodiments described herein, connected to standard hubs and plungers, after the plunger has been fully pressed to expel fluid loaded within hub. This emulates, for example, amount of “dead” fluid which is lost after administering a vaccine.

### Materials

Inventors created two syringes.

A first syringe was created by connecting the needle hub assemblies based on embodiments described herein, to standard 1 mL Luer Slip Tip barrel and plunger.

A second syringe was created by connecting a standard needle hub assembly to the standard 1 mL syringe Luer Slip Tip.

The first syringe was filled with colored water.

The second syringe was filled with colored water.

### Methods

### First Syringe (Including Hub Assembly Based on Embodiments Described Herein):

The weight of the first syringe filled with water was 2.4349 grams.

Following the weighing of the first syringe filled with water, the plunger was pressed to its maximal displacement to expel as much water as possible. The weight of the first syringe with retained water was weighed. The weight of the first syringe with retained was 2.4137 grams.

The total amount of dead space (i.e., retained water) of the syringe was calculated as 2.4349−2.4137=0.0212 grams, which is 0.0212 mL.

The total dead space of the standard 1 mL Luer Slip Tip barrel and plunger is known to be 0.016 mL.

The total dead space of the needle hub assembly based on embodiments described herein was calculated as 0.0212−0.016=0.0052 mL.

### Second Syringe (Includes Standard Components):

The weight of the second syringe filled with water was 2.4036 grams.

Following the weighing of the second syringe filled with water, the plunger was pressed to its maximal displacement to expel as much water as possible. The weight of the second syringe with retained water was weighed. The weight of the second syringe with retained was 2.3684 grams.

The total amount of dead space (i.e., retained water) of the syringe was calculated as 2.4036−2.3684=0.0352 grams, which is 0.0352 mL.

The total dead space of the standard 1 mL Luer Slip Tip barrel and plunger is known to be 0.016 mL.

The total dead space of the standard needle hub assembly was calculated as 0.0352−0.016=0.0192 mL.

### CONCLUSION

The volume retained by the needle hub assembly based on embodiments described herein was measured to be 0.0052 mL or 5 microliter, which is significantly less than the volume retained by the standard needle hub assemble which was measured to be 0.0192 mL or 19 microliters.

Using the needle hub assembly based on embodiments described herein saves up to about 0.0014 mL or 14 microliters (i.e., ie 0.0192 mL-0.0052 mL) of fluid in comparison to using standard components of standard syringes (i.e., standard hub, standard barrel, and standard plunger).

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

What is claimed is:

1. A needle hub assembly (102) for connecting to a barrel with plunger for assembling a syringe, comprising:
a cylindrical depression (104) in a proximal end portion (106) of the needle hub assembly, the cylindrical depression having a flat bottom (108) perpendicular to a longitudinal axis (110) of the needle hub assembly,
wherein the cylindrical depression is shaped for connecting to a nozzle of the barrel and for contacting between the flat bottom and a distal end of the nozzle surrounding an opening of the nozzle when the nozzle is disposed within the cylindrical depression;
a lumen (112) extending from the flat bottom of the cylindrical depression to a distal end portion (114) of the needle hub assembly; and
a cannula (116) having a proximal cannula portion (118) located within the lumen and a distal cannula portion (134) extending distally from the distal end portion of the needle hub assembly.

2. The needle hub assembly of claim 1, wherein a depth of the cylindrical depression measured parallel to the longitudinal axis is greater than 7 millimeters (mm).

3. The needle hub assembly of claim 1, wherein an inner surface of the cylindrical depression extending from the flat bottom to a depth 120 away from an edge (122) of the proximal end portion, is sized and shaped to contact and fluidly seal an exterior surface of the nozzle of the barrel when the barrel is disposed within the cylindrical depression.

4. The needle hub assembly of claim 1, wherein the inner surface of the cylindrical depression is sized and/or shaped for compliance with ISO 80369-7.

5. The needle hub assembly of claim 1, wherein the needle hub assembly is sized and/or shaped for compliance with ISO 594-1.

6. The needle hub assembly of claim 1, wherein the inner surface of the cylindrical depression is set to a 6% Luer tap.

7. The needle hub assembly of claim 1, further comprising a pair of flares (124A-B) extending outwardly from opposite locations of an external surface of an end of the proximal end portion of the needle hub assembly, wherein a length from an end of a first flare (124A) to an end of a second flare (124B) perpendicular to the longitudinal axis is about 7.8 millimeters and a diameter of the first end portion excluding the pair of flares is about 6.7 mm.

8. The needle hub assembly of claim 1, wherein the cylindrical depression is sized and shaped for fitting Luer slip and Luer Lock Tip devices.

9. The needle hub assembly of claim 1, wherein the cylindrical depression and cannula are sized and shaped to enclose a volume of about 0.0052 milliliters (mL) when a low dead space plunger is fully plunged into the barrel connected to the cylindrical depression.

10. The needle hub assembly of claim 1, wherein the lumen comprises a proximal lumen portion (126) having a constant internal diameter along a length thereof and a distal lumen portion (128) having a tapered diameter increasing in diameter from a junction (130) with the proximal lumen portion towards an opening at the distal end portion of the needle hub assembly, the proximal lumen portion having an opening (132) into the cylindrical depression.

11. The needle hub assembly of claim 10, wherein the proximal cannula portion is disposed within the distal lumen portion, wherein at the junction the cannula is in continuity with the distal lumen portion, wherein the cannula has a constant inner diameter along a length thereof matching a constant inner diameter of the distal lumen portion, thereby forming a fluid flowing channel with continuous constant inner diameter for fluid flowing from the barrel via the cylindrical depression via the proximal lumen portion and through the cannula.

12. The needle hub assembly of claim 11, wherein the cannula is not disposed within the proximal lumen portion.

13. The needle hub assembly of claim 11, wherein an adhesive is disposed within a space (138) formed between an exterior surface of the proximal cannula portion disposed within the lumen and an interior surface of the tapered distal lumen portion.

14. The needle hub assembly of claim 13, wherein the adhesive extends externally from the space, forming a cone and/or convex shape (140) having a wider base connected to an end surface of the distal end portion of the needle hub assembly perpendicular to the longitudinal axis, wherein the cannula extends through an interior of the cone and/or convex shape of the extending adhesive.

15. The needle hub assembly of claim 1, further comprising a support element (140) having a wider base connected to an end surface of the distal end portion of the needle hub assembly perpendicular to the longitudinal axis, wherein the cannula extends through an interior of the support element.

16. The needle hub assembly of claim 15, wherein the support element comprises an adhesive that supports a portion of the cannula extending past the distal end portion of the needle hub assembly by connecting the cannula extending through the interior of the support element with the end surface of the distal end portion of the needle hub assembly.

17. The needle hub assembly of claim 1, wherein the cannula is made using medical silicon.

18. The needle hub assembly of claim 1, wherein the cannula is lubricated by medical silicon fluid.

19. The needle hub assembly of claim 1, wherein the cannula is made from materials compliant with ISO 9626.

20. The needle hub assembly of claim 1, wherein a bevel angle (142) of the cannula comprises a triple point.
